(19) United States
(12) Patent Application Publication
(10) Pub. No.: US 2016/0106367 A1
(43) Pub. Date: Apr. 21, 2016

(54) WEARABLE HEALTH SENSOR

(71) Applicant: SERAPHIM SENSE LTD., Tel Aviv (IL)

(72) Inventors: Eugene Jorov, Tel Aviv (IL); Amir Shlomovich, Nordiya (IL)

(21) Appl. No.: 14/891,969

(22) PCT Filed: Jun. 11, 2014
(86) PCT No.: PCT/IB2014/062133

§ 371 Date: Nov. 18, 2015

Related U.S. Application Data
(60) Provisional application No. 61/841,440, filed on Jul. 1, 2013.

(51) Int. Cl. A61B 5/00; A61B 5/1455; A61B 5/024

(52) U.S. Cl. CPC A61B 5/6824; A61B 5/6831; A61B 5/74; A61B 5/02438; A61B 5/02416; A61B 5/7278; A61B 5/14551; A61B 2562/0204; A61B 2562/0219; A61B 2560/0214

(57) ABSTRACT

A monitoring device (24) includes a band (32), configured to fit around an appendage of a human subject (22), and one or more sensors (46, 58, 60, 62) fixed to the band and configured to sense physiological parameters of the human subject. In one embodiment, at least one visible light source (82) is fixed to the band. A controller (64) is fixed to the band and is coupled to receive signals from the sensors and to actuate the at least one visible light source in response to a state of the device. At least one flexible light guide (40) is embedded in an outer surface of the band while extending around at least a part of a circumference of the band when fit around the appendage, and is coupled to receive light from the visible light source and to emit the light from the outer surface in a bright stripe along a length of the light guide.

### CROSS-REFERENCE TO RELATED APPLICATION

This application claims the benefit of U.S. Provisional Patent Application 61/841,440, filed Jul. 1, 2013, which is incorporated herein by reference.

### FIELD OF THE INVENTION

The present invention relates generally to health monitoring, and particularly to wristbands containing integrated sensors.

### BACKGROUND

Wearable electronic monitors have become popular not only for medical use, but also for personal health monitoring, particularly during sports activities. For example, the MIO Alpha is a wristwatch-shaped device that measures heart rate. The device has a wireless Bluetooth® interface for connecting to applications on a computer or smartphone. As another example, the Basis wristwatch monitor includes an optical blood flow sensor, an accelerometer, a perspiration monitor, and a skin temperature sensor.

Various wristwatch-type monitors have been described in the patent literature. For example, U.S. Patent Application Publication 2012/0226112 describes real-time, noninvasive health and environmental monitors that include a plurality of compact sensors integrated within small, low-profile devices. Physiological and environmental data is collected and wirelessly transmitted into a wireless network, where the data is stored and/or processed. This information is then used to support a variety of methods, such as clinical trials, marketing studies, biofeedback, entertainment, and others.

As another example, U.S. Pat. Nos. 8,012,097 and 8,579,827 describe a monitoring device for monitoring the vital signs of a user. The device preferably includes a band worn on a user's wrist, arm or ankle, with an optical sensor, an accelerometer and processor. The optical sensor preferably comprises a photodetector and a plurality of light emitting diodes. A sensor signal from the optical sensor is processed with a filtered accelerometer output signal from the accelerometer to create a filtered vital sign signal used to generate a real-time vital sign for a user. The device may include a short-range wireless transmitter, which receives information from the processor and transmits this information in the form of a packet through an antenna. An external laptop computer or hand-held device features a similar antenna coupled to a matched wireless, short-range receiver that receives the packet.

Yet another example is provided by U.S. Patent Application Publication 2006/0122520, which describes a medical device that measures vital signs (e.g., blood pressure, pulse oximetry, and heart rate) from a patient using at least two optical modules. Each optical module typically features two light sources (red, infrared) and a photodetector. Both optical modules are configured to measure time-dependent signals describing the patient's flowing blood. A processor analyzes the time-dependent signals to determine the patient's vital signs.

The disclosures of the publications cited above are incorporated herein by reference for purposes of the information that they provide on implementation of vital sign measurement and monitoring, but are not to serve as a basis for claim construction in the present patent application.

### SUMMARY

Embodiments of the present invention that are described hereinbelow provide a wrist-mounted health monitor with enhanced features.

There is therefore provided, in accordance with an embodiment of the present invention, a monitoring device, which includes a band, configured to fit around an appendage of a human subject, and one or more sensors fixed to the band and configured to sense physiological parameters of the human subject. At least one visible light source is fixed to the band. A controller, which is fixed to the band, is coupled to receive signals from the one or more sensors, and is configured to actuate the at least one visible light source in response to a state of the device. At least one flexible light guide, embedded in an outer surface of the band while extending around at least a part of a circumference of the band when fit around the appendage, is coupled to receive light from the visible light source and to emit the light from the outer surface in a bright stripe along a length of the light guide.

In a disclosed embodiment, the at least one flexible light guide includes at least first and second light guides, which are respectively embedded in opposing first and second edges of the band, and the at least one visible light source is embedded inside the band.

In one embodiment, the controller is configured to establish the state of the device in response to the signals from at least one of the sensors and to actuate the at least one visible light source in response to the established state. The controller may be configured to actuate the at least one visible light source in response to a pulse rate of the human subject.

There is also provided, in accordance with an embodiment of the present invention, a monitoring device, which includes a band, configured to fit around an arm of a human subject, and a sensing module, which is embedded within the band so as to contact a skin surface of the human subject wearing the device. The sensing module includes a light detector; first and second light sources, configured to emit light of a first wavelength, at respective first and second locations alongside the light detector; and third and fourth light sources, configured to emit light of a second wavelength, different from the first wavelength, at respective third and fourth locations alongside the light detector. A controller, which is fixed to the band, is coupled to actuate the first, second, third and fourth light sources in alternation, to receive signals from the light detector in response to the emitted light, and to compare the signals in order to extract vital signs of the subject.

In some embodiments, the vital signs extracted by the controller include a pulse rate. The sensing module may include an acoustic sensor and an accelerometer, wherein the controller is configured to receive and apply respective outputs of the acoustic sensor and the accelerometer together with the signals from the light detector in extracting the pulse rate.

Additionally or alternatively, the vital signs extracted by the controller may include a blood oxygen saturation level. In one embodiment, the first and second light sources emit green light, and the third and fourth light sources emit blue light.

Typically, the first and third light sources are disposed on a first side of the light detector, and the second and fourth light sources are disposed on a second side of the light detector, opposite the first side.

There is additionally provided, in accordance with an embodiment of the present invention, a monitoring device, which includes a band, including a flexible dielectric material, which is configured to fit around an appendage of a human subject. A sensing circuit is embedded in a first area the band and configured to sense physiological parameters of the human subject. At least one battery is embedded in at least one second area of the band. At least one resilient, conductive strip, which is pre-formed in a curved shape to fit around the appendage, is embedded in the band, and is connected to the sensing circuit and the at least one battery so as to provide power from the at least one battery to the sensing circuit.

In a disclosed embodiment, the at least one battery includes first and second batteries, disposed within the band on opposing sides of the sensing circuit, and the at least one resilient, conductive strip includes first and second strips, which respectively connect the first and second batteries to the sensing circuit. Typically, each of the first and second strips extends around more than 90° along a circumference of the band.

In some embodiments, the device includes a clasp attached to the band for closing the band around the appendage, wherein the clasp includes a conductive pin, which is electrically connected to the at least one battery and is connectable to a power source external to the device in order to charge the at least one battery.

There is further provided, in accordance with an embodiment of the present invention, a monitoring device, which includes a band, configured to fit around an appendage of a human subject, and a sensing circuit fixed to the band and configured to sense physiological parameters of the human subject. At least one battery is embedded in the band and coupled to provide electrical power to the sensing circuit. A clasp is attached to the band for closing the band around the appendage, and includes at least one conductive pin, which is electrically connected to the at least one battery and is connectable to a power source external to the device in order to charge the at least one battery.

In a disclosed embodiment, the at least one battery includes first and second batteries, disposed respectively on opposing sides of the clasp, and the clasp includes first and second pairs of conductive pins, which are respectively connected to the first and second batteries. The clasp may include an elastic polymer piece having openings formed therein to fit over and secure both pairs of the conductive pins.

There is moreover provided, in accordance with an embodiment of the present invention, a monitoring device, which includes a band, configured to fit around an appendage of a human subject and having opposing, first and second ends, and a sensing circuit fixed to the band and configured to sense physiological parameters of the human subject. A clasp, which is attached to the band for closing the band around the appendage, includes first and second pairs of pins, which protrude in a transverse direction from the first and second ends of the band, respectively, and an elastic polymer piece having transverse openings formed therein to fit over and secure both pairs of the pins, thereby closing the band circumferentially around the appendage.

In a disclosed embodiment, the device includes set of polymer pieces having different, respective lengths in a direction circumferential to the band for fitting appendages of different sizes, wherein the elastic polymer piece that closes the band is selected from the set.

There is furthermore provided, in accordance with an embodiment of the present invention, a method for monitoring, which includes providing a monitoring device, which includes a band, configured to fit around an appendage of a human subject and having opposing, first and second ends, with end pieces protruding from the first and second ends of the band, respectively, and which includes a sensing circuit fixed to the band and configured to sense physiological parameters of the human subject. A set of polymer pieces is provided, having openings formed therein to fit over and secure the end pieces of the band and having different, respective lengths in a direction circumferential to the band. One of the polymer pieces is chosen, having a respective length appropriate to a size of the appendage of the human subject. The chosen one of the polymer pieces is fitted over the end pieces in order to close the band.

In a disclosed embodiment, the end pieces include conductive terminals, which are electrically connected to a battery in the monitoring device, and the method includes coupling the end pieces to a power source in order to charge the battery.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a health monitoring system, in accordance with an embodiment of the present invention;

FIG. 2 is a schematic, pictorial illustration of a health monitoring device, in accordance with an embodiment of the present invention;

FIGS. 3A and 3B are schematic internal views of a health monitoring device, in accordance with an embodiment of the present invention;

FIG. 4A is a schematic, pictorial illustration of a clasp for health monitoring device, in accordance with an embodiment of the present invention; and

FIG. 4B is a schematic detail view of a fastening and charging mechanism in a health monitoring device, in accordance with an embodiment of the present invention.

### DETAILED DESCRIPTION OF EMBODIMENTS

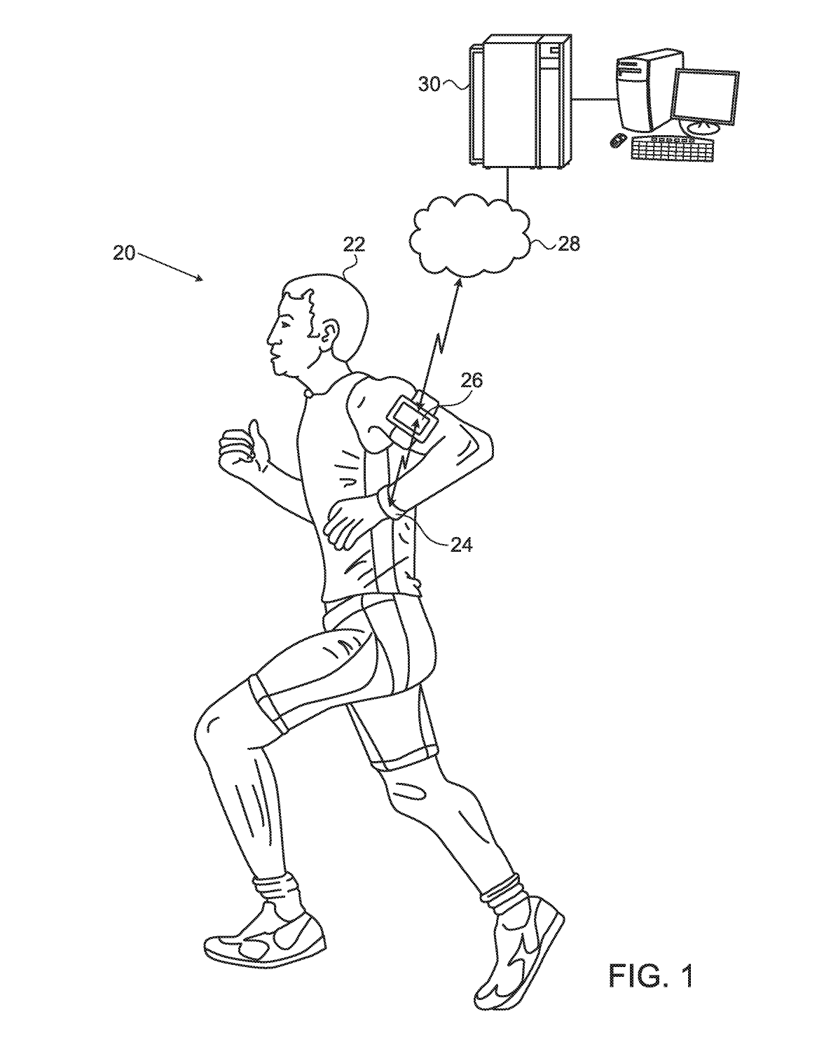

FIG. 1 is a schematic, pictorial illustration of a health monitoring system 20, in accordance with an embodiment of the present invention. In the pictured embodiment, a human subject 22 wears a monitoring device 24 in the form of a band around his wrist. Alternatively, the band may be configured to fit around another part of the arm or any other suitable appendage of the subject's body. As described in greater detail hereinbelow, device 24 comprises one or more sensors fixed to the band, which sense physiological parameters of subject 22, such as heart rate, blood oxygen saturation, temperature, and body motion, for example. Subject 22 can wear and use device 24 intermittently, such as during sports activities, or continuously—even twenty-four hours a day, and seven days a week.

Monitoring device 24 comprises a wireless communication interface, such as a Bluetooth transceiver, which communicates over the air with a computing device 26 in order to transmit physiological data and receive instructions. In the pictured example, computing device 26 is a mobile device, such as a smartphone or personal digital assistant. Alternatively, in other applications of monitoring device 24, computing device 26 may comprise a portable or desktop computer or any other such device with suitable computing and communication capabilities.

Computing device 26 typically runs a program that processes the data transmitted by monitoring device 24 in order to compute, store and display monitoring results. These results may, for example, provide feedback to subject 22 on his performance during or after sports training, or they may be made available to a medical or physical training professional in order to assess the condition of the subject. For this latter purpose particularly (but not only for this purpose), computing device 26 may communicate over a network 28 with a server 30 or other computer, which may store, analyze, extract statistics from, or perform any other desired processing of the data provided by device 24.

In some embodiments, monitoring device 24 may have an open application program interface (API), which enables programs running on computing device 26 to control the functions of monitoring device 24 and select the data to receive and process from the monitoring device. This API enables developers to write application programs that use the data provided by monitoring device 24 in different ways, for purposes such as sports training, sleep monitoring, and medical diagnosis and alerts. For this latter purpose particularly, the API may provide access not only to processed data (such as heart rate, blood oxygen level, and motion) that is provided by the processor in monitoring device 24, but also to the raw output signals of the sensors. The inventors envision that on the basis of the open API, third-party developers will be able to independently develop a range of different applications for computing device 26 that make use of the capabilities of monitoring device 24 and will offer these applications for download to suitable computing devices (such as smart phones, computers, and fitness gear) of users of the monitoring device.

The figures that follow show details of monitoring device 24, and specifically illustrate certain novel features of the device. For concreteness and clarity of illustration, the figures show all of these features as elements of the same device (and indeed, this combination of features makes the device particularly useful, comfortable and robust). In alternative embodiments, however, which are not shown in the figures, these features may be implemented individually, independently of one another, or in various sub-combinations. All such implementations are considered to be within the scope of the present invention.

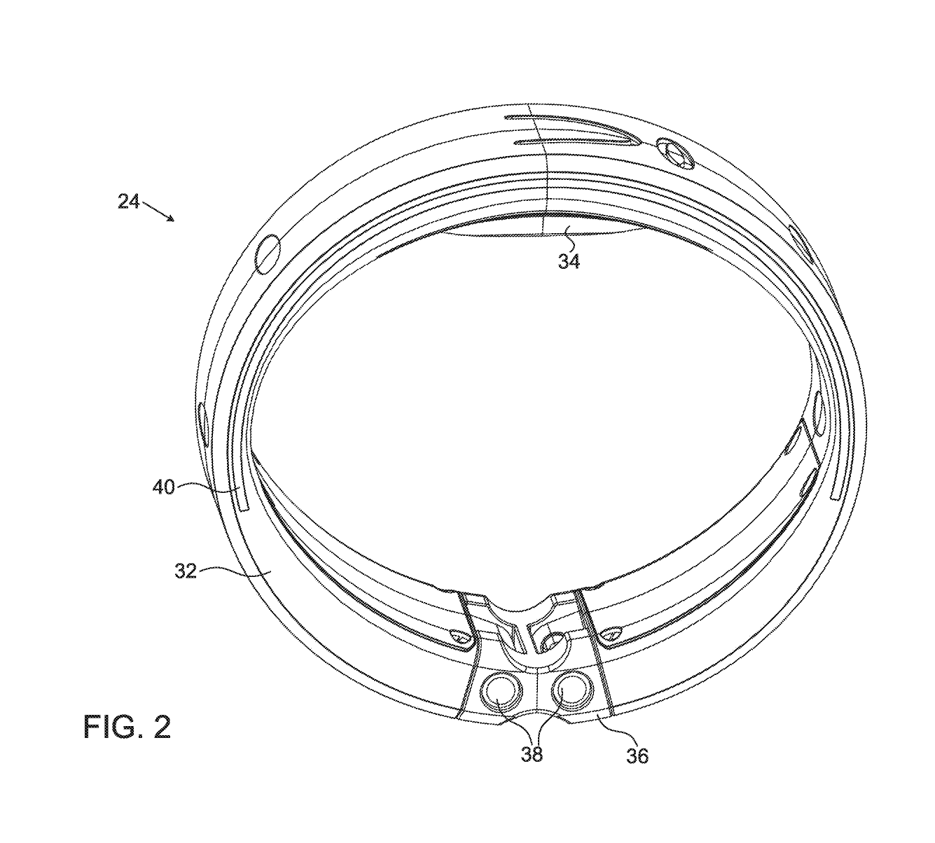

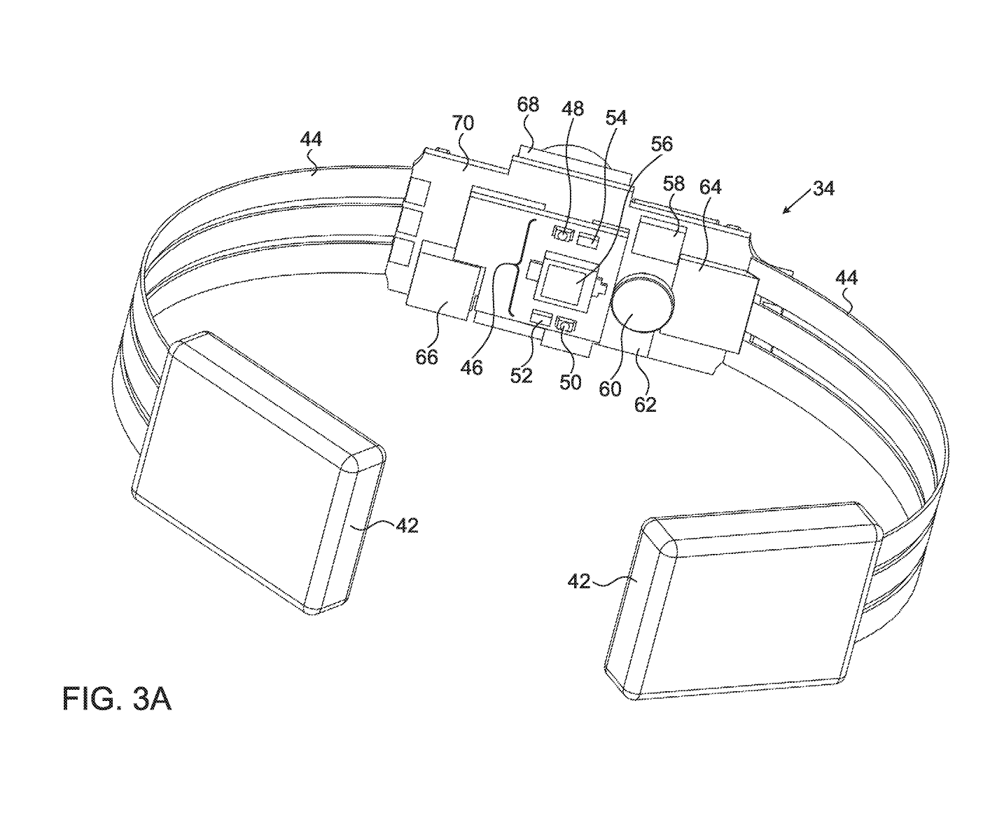

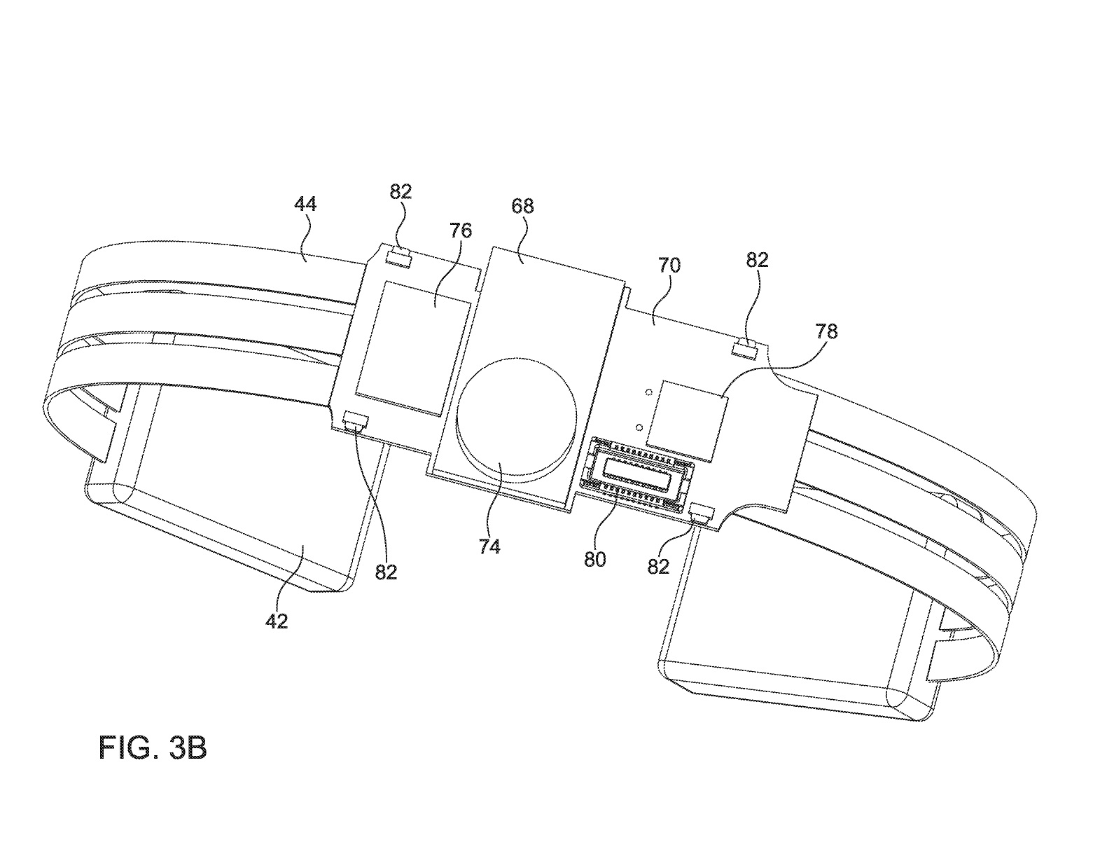

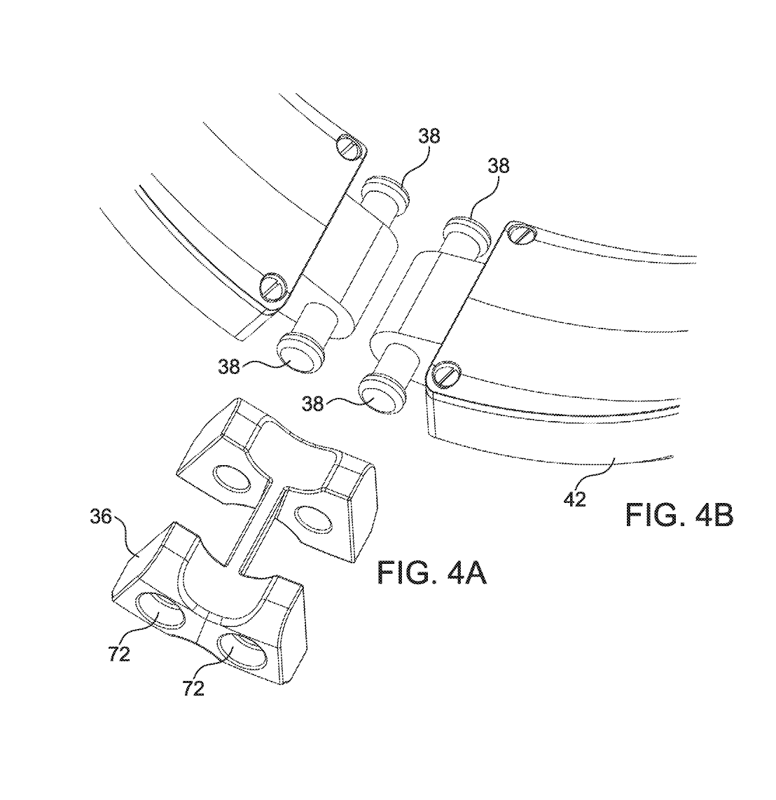

FIG. 2 is a schematic, pictorial illustration of monitoring device 24, in accordance with an embodiment of the present invention. Device 24 comprises a band 32, which fits around the wrist (or another appendage) of subject 22. Band 32 may comprise any suitable dielectric material, such as a flexible, molded polymer. A capsule 34 fixed inside band 32 contains sensors, which sense physiological parameters of the subject, as described particularly with reference to FIG. 3A. The location of capsule 34 is advantageous in making firm contact with the subject's skin while the subject wears device 24. Alternatively or additionally, however, sensors may be embedded in or otherwise fixed to band 32 at other locations. A clasp 36 fits over pins 38 to fasten band 32 firmly around the subject's wrist. Features of these latter elements of device 24 are described further hereinbelow with reference to FIGS. 4A and 4B.

At least one flexible light guide 40 is embedded in the outer surface of band 32 and extends at least part way around the circumference of the band when fit around the wrist. One or more light sources inside band 32 or capsule 34 (as shown in FIG. 3B) emit visible light, which is coupled into light guide 40 from within band 32 and then emitted from the outer surface of the band in a bright stripe along the length of the light guide. For this purpose, the light guide may comprise any suitable transparent material, typically a plastic material, such as an acrylic or polycarbonate polymer. Although light guide 40 appears in FIG. 2 as a single unit, in practice device 24 may contain two or more light guides, which may be embedded in one or both sides of band 32 (i.e., on the side facing into the page of FIG. 2, in addition to the side that is visible in the figure).

A controller (shown in FIG. 3A) actuates the light sources in device 24 to feed light to light guide 40 in response to the state of the device. The “state” of the device in this context should be understood broadly to refer to any sort of state associated with the operation of device 24, including not only the operational status of the circuits in device 24, but also to state parameters derived from physiological measurements made by the device or from communications with computing device 26. In other words, the controller may establish the state in response to signals from one or more of the sensors in capsule 34, and then actuate the visible light source in response to the established state.

For example, the controller may actuate one or more of the visible light sources in response to the measured pulse rate of subject 22. The light sources may then cause light guide 40 to flash in synchronization with the pulse rate, and/or possibly to change the intensity or color of the emitted light based on the pulse rate and/or other parameters. Other uses of the light guides in device 24 in communicating information to subject 22 will be apparent to those skilled in the art (and may be controlled by applications running on computing device 26, using the above-mentioned API), and are considered to be within the scope of the present invention. The use of light guides 40 in communicating with subject 22 is advantageous, by comparison with conventional sorts of indicator lamps, in the high visibility and durability that the light guides provide.

FIGS. 3A and 3B are schematic internal views of device 24, in accordance with an embodiment of the present invention. These figures show the internal electronic and structural components of device 24, without the encapsulation that is normally provided by band 32. While capsule 34 is located at the center of the band, batteries 42 are embedded in other areas of the band, typically alongside clasp 36. Although device 24 may be powered by only a single battery, the dual-battery configuration shown in the figures, with the batteries disposed within the band on opposing sides of the sensing circuits in capsule 34, is advantageous in providing both greater power and a better-balanced mass distribution around band 32.

Batteries 42 are connected to the circuits in capsule 34 by one or more resilient, conductive strips 44, which are pre-formed in a curved shape to fit around the wrist of the subject and are embedded in band 32. Strips 44 may comprise copper, for example, or any other suitable metal with sufficient flexibility to allow band 32 to fit different wrist shapes. These strips perform the dual functions of providing power from batteries 42 to the sensing circuits and providing mechanical rigidity to band 32, so that the band will fit stably and comfortably around the subject's wrist while maintaining good contact between the sensors and the subject's skin. For this purpose, each of strips 44 typically extends around 90° or more of a circumference of band 32.

The pre-formed semi-rigid nature of conductive strips 44 enables precise positioning of the strips inside an injection mold during manufacturing (as opposed to a flexible flat cable, for example). The ability to control exact placement of the electronics and interconnecting parts inside the injection mold, with the support of strips 44, is crucial to the manufacturing process. Uncontrolled placement might lead to internal device elements breaking the outer surface of the wristband and thus result in loss of yield.

Among the sensors within capsule 34, as illustrated in FIG. 3A, device 24 comprises an optical sensing module 46, which can be used to measure pulse rate and blood oxygen saturation. Module 46 is embedded within band 32 in capsule 34 so as to contact the skin surface of the human subject wearing the device. The module comprises a light detector 56, such as a silicon photodiode, and light sources 48, 50, 52 and 54, which typically comprise light-emitting diodes (LEDs). Light sources 48 and 50 emit light of one selected wavelength, while light sources 52 and 54 emit light of a different wavelength from that of light sources 48 and 50.

For example, to enable measurement of blood oxygen saturation using methods of pulse oximetry that are known in the art, light sources 48 and 50 may emit red light, while light sources 52 and 54 emit infrared light. A controller 64 actuates light sources 48, 50, 52 and 54 in alternation, receive signals from light detector 56 in response to the emitted light, and compares the signals in order to extract vital signs of the subject, such as the pulse rate and oxygen saturation level. Controller 64 typically comprises a microprocessor with suitable interfaces and software code. The use of redundant pairs of light sources 48/50 and 52/54 in the sort of crisscross placement that is shown in FIG. 3A is useful in enhancing the reliability of measurements even during active motion of the subject and in the presence of sweat and dirt that may come between capsule 34 and the subject's skin.

Other arrangements of the light sources and choices of wavelengths are also possible. For example, in an alternative embodiment, light sources 48 and 50 may emit green light, while light sources 52 and 54 emit blue light. The inventors have found that this unconventional choice of illumination wavelengths can provide a more accurate reading of blood oxygen saturation under some circumstances. As another alternative, other pairings of the light sources may be used; for example, light sources 48 and 54 may emit one wavelength, while light sources 50 and 52 emit the other.

For accurate measurement of cardiac function, capsule 34 may also contain an acoustic sensor, such as a microphone 58, which senses acoustical signals due to the pulsatile blood flow in the wrist of subject 22. Processor 64 may combine the acoustical information provided by microphone 58 with the optical measurements made by light sensor 56 in order to provide a more accurate and robust measurement of the heart rate. Details of this technique are described in the above-mentioned provisional patent application. An accelerometer 62, which senses motion of device 24 and hence of the subject's arm or other appendage, can be used to further enhance the robustness of measurement of the pulse rate by enabling motion artifacts to be detected and filtered out.

In addition, capsule 34 may contain a temperature sensor 60, which is likewise mounted in contact with the subject's skin. The various sensors, including sensing module 46, microphone 58, accelerometer 62 and temperature sensor 60, are typically mounted on a printed circuit board 70, which is embedded in band 32. Other sensing and control components may likewise be mounted on board 70 and operate under the control of controller 64, including, for example:
A speaker 66, which outputs audible tones and other audio signals.
A Bluetooth transceiver 68 (typically, though not necessarily, Bluetooth Low Energy) or other short-range radio link.
A motor 74, which causes capsule 34 to vibrate in order to provide haptic signals to subject 22.
A memory 76 for data and program code.
A switch 78, which may be operated by subject 22 to turn device 24 on and off (typically by pressing externally on band 32 in the location of the switch), and possibly to invoke other user-controlled functions, such as hard reset when needed (by pressing and holding the switch for an extended period).
A radio-frequency identification (RFID) tag.
Visible light sources 82, such as LEDs. These light sources couple light into light guides 40 when actuated by controller 64 and thus carry out the signaling functions that were described above.

FIGS. 4A and 4B are schematic, pictorial illustrations showing details of a fastening and charging mechanism in monitoring device 24, in accordance with an embodiment of the present invention. FIG. 4A shows clasp 36, while FIG. 4B shows pins 38 that are used to secure the clasp in order to close band 32 around the wrist. End pieces, in the form of two pairs of pins 38, are connected to the two opposing ends of band 32. Pins 38 in this embodiment protrude in a transverse direction from the ends of the band (i.e., in a direction perpendicular to the circumference of the band). Clasp 36 is made from a piece of elastic polymer, having transverse openings 72 that fit over and secure both pairs of pins 38, thereby closing band 32 circumferentially around the wrist.

To fit device 24 to different subjects, of different wrist sizes, band 32 may be provided with a set of clasps 36 having different, respective lengths in the circumferential direction. The subject, or else a sales or service person supplying the device, selects one of the clasps from the set that best suits the size of the subject's wrist. The chosen clasp is then fitted over the end pieces in order to close the band. In contrast to watchbands and other sorts of wristbands that are known in the art, no special tools or skills are needed in order to make the length adjustment, and the absence of a buckle (which could otherwise be used for length adjustment) enhances the comfort and reliability of device 24 for the subject wearing it. Although FIGS. 4A and 4B show a particular design of the end pieces and elastic polymer clasp, other designs may similarly be used in this manner to fit different subjects, and are considered to be within the scope of the present invention.

The design of the clasp end pieces that is shown in FIG. 4B has the additional advantage that pins 38 can be made of a conductive material, such as a suitable metal, and serve as electrical terminals connected to batteries 42. Specifically, when device 24 is removed from the wrist, and clasp 36 is removed from pins 38, the pins are able to plug into a dedicated external power source (not shown in the figures) in order to charge the batteries 42. In the pictured embodiments, both pairs of pins 38 can be used to charge the respective batteries 42 at each end of band 32. Alternatively, only one pair of pins may be electrically connected, either at the same end or opposite ends of the band. Further alternatively or additionally, each of the pictured pins may actually comprise a pair of terminals (positive and negative), which are separated from one another by built-in insulation.

Light guide 40 may be used to indicate various charging phases to the user. For example, fast blinking or pulsating light may be used to indicate low battery level. The frequency of the blinking or pulsating light may be decreased as the charging process nears completion. To indicate the battery is full, the light may be turned on constantly.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A monitoring device, comprising:
a band, configured to fit around an appendage of a human subject;
one or more sensors fixed to the band and configured to sense physiological parameters of the human subject;
at least one visible light source fixed to the band;
a controller, which is fixed to the band and is coupled to receive signals from the one or more sensors, and which is configured to actuate the at least one visible light source in response to a state of the device; and
at least one flexible light guide embedded in an outer surface of the band while extending around at least a part of a circumference of the band when fit around the appendage, and coupled to receive light from the visible light source and to emit the light from the outer surface in a bright stripe along a length of the light guide.

2. The device according to claim 1, wherein the at least one flexible light guide comprises at least first and second light guides, which are respectively embedded in opposing first and second edges of the band.

3. The device according to claim 1, wherein the at least one visible light source is embedded inside the band.

4. The device according to claim 1, wherein the controller is configured to establish the state of the device in response to the signals from at least one of the sensors and to actuate the at least one visible light source in response to the established state.

5. The device according to claim 4, wherein the controller is configured to actuate the at least one visible light source in response to a pulse rate of the human subject.

6. A monitoring device, comprising:
a band, configured to fit around an arm of a human subject;
a sensing module, which is embedded within the band so as to contact a skin surface of the human subject wearing the device, and which comprises:
a light detector;
first and second light sources, configured to emit light of a first wavelength, at respective first and second locations alongside the light detector; and
third and fourth light sources, configured to emit light of a second wavelength, different from the first wavelength, at respective third and fourth locations alongside the light detector; and
a controller, which is fixed to the band and is coupled to actuate the first, second, third and fourth light sources in alternation, to receive signals from the light detector in response to the emitted light, and to compare the signals in order to extract vital signs of the subject.

7. The device according to claim 6, wherein the vital signs extracted by the controller comprise a pulse rate.

8. The device according to claim 7, wherein the sensing module comprises an acoustic sensor and an accelerometer, and wherein the controller is configured to receive and apply respective outputs of the acoustic sensor and the accelerometer together with the signals from the light detector in extracting the pulse rate.

9. The device according to claim 6, wherein the vital signs extracted by the controller comprise a blood oxygen saturation level.

10. The device according to claim 9, wherein the first and second light sources emit green light, and the third and fourth light sources emit blue light.

11. The device according to claim 6, wherein the first and third light sources are disposed on a first side of the light detector, and the second and fourth light sources are disposed on a second side of the light detector, opposite the first side.

12. A monitoring device, comprising:
a band, comprising a flexible dielectric material, which is configured to fit around an appendage of a human subject;
a sensing circuit embedded in a first area the band and configured to sense physiological parameters of the human subject;
at least one battery embedded in at least one second area of the band; and
at least one resilient, conductive strip, which is pre-formed in a curved shape to fit around the appendage and is embedded in the band, and which is connected to the sensing circuit and the at least one battery so as to provide power from the at least one battery to the sensing circuit.

13. The device according to claim 12, wherein the at least one battery comprises first and second batteries, disposed within the band on opposing sides of the sensing circuit, and wherein the at least one resilient, conductive strip comprises first and second strips, which respectively connect the first and second batteries to the sensing circuit.

14. The device according to claim 13, wherein each of the first and second strips extends around more than 90° along a circumference of the band.

15. The device according to claim 13, and comprising a clasp attached to the band for closing the band around the appendage, wherein the clasp comprises a conductive pin, which is electrically connected to the at least one battery and is connectable to a power source external to the device in order to charge the at least one battery.

16. A monitoring device, comprising:
a band, configured to fit around an appendage of a human subject;
a sensing circuit fixed to the band and configured to sense physiological parameters of the human subject;
at least one battery embedded in the band and coupled to provide electrical power to the sensing circuit; and
a clasp, which is attached to the band for closing the band around the appendage, and which comprises at least one conductive pin, which is electrically connected to the at least one battery and is connectable to a power source external to the device in order to charge the at least one battery.

17. The device according to claim 16, wherein the at least one battery comprises first and second batteries, disposed respectively on opposing sides of the clasp, and wherein the clasp comprises first and second pairs of conductive pins, which are respectively connected to the first and second batteries.

18. The device according to claim 17, wherein the clasp comprises an elastic polymer piece having openings formed therein to fit over and secure both pairs of the conductive pins.

19. A monitoring device, comprising:
a band, configured to fit around an appendage of a human subject and having opposing, first and second ends;
a sensing circuit fixed to the band and configured to sense physiological parameters of the human subject; and
a clasp, which is attached to the band for closing the band around the appendage, and which comprises:
first and second pairs of pins, which protrude in a transverse direction from the first and second ends of the band, respectively; and
an elastic polymer piece having transverse openings formed therein to fit over and secure both pairs of the pins, thereby closing the band circumferentially around the appendage.

20. The device according to claim 19, and comprising a set of polymer pieces having different, respective lengths in a direction circumferential to the band for fitting appendages of different sizes, wherein the elastic polymer piece that closes the band is selected from the set.

21. A method for monitoring, comprising:
providing a monitoring device, which comprises a band, configured to fit around an appendage of a human subject and having opposing, first and second ends, with end pieces protruding from the first and second ends of the band, respectively, and which comprises a sensing circuit fixed to the band and configured to sense physiological parameters of the human subject;
providing a set of polymer pieces having openings formed therein to fit over and secure the end pieces of the band and having different, respective lengths in a direction circumferential to the band;
choosing one of the polymer pieces having a respective length appropriate to a size of the appendage of the human subject; and
fitting the chosen one of the polymer pieces over the end pieces in order to close the band.

22. The method according to claim 21, wherein the end pieces comprise conductive terminals, which are electrically connected to a battery in the monitoring device, and wherein the method comprises coupling the end pieces to a power source in order to charge the battery.
